(19) United States
(12) Patent Application Publication
(10) Pub. No.: US 2023/0190984 A1
(43) Pub. Date: Jun. 22, 2023

(54) AIR PURIFYING DEVICE

(71) Applicant: AIRTURA LIMITED, Milton Keynes, Buckinghamshire (GB)

(72) Inventor: David WHITEHEAD, Milton Keynes (GB)

(21) Appl. No.: 17/927,149

(22) PCT Filed: May 21, 2021
(86) PCT No.: PCT/GB2021/051239

§ 371 Date: Nov. 22, 2022

(30) Foreign Application Priority Data
May 22, 2020 (GB) 2007696.4
Aug. 4, 2020 (GB) 2012074.7
Dec. 17, 2020 (GB) 2020023.4

(51) Int. Cl. A61L 9/20; A61L 9/12; A61L 9/22; A61L 2/20; A61L 2/26; B01J 21/06; B01J 23/06; B01J 35/00

(52) U.S. Cl. CPC A61L 9/205; A61L 9/12; A61L 9/22; A61L 2/202; A61L 2/26; B01J 21/063; B01J 23/06; B01J 35/004; A61L 2209/12; A61L 2209/14; A61L 2209/16; A61L 2101/02

(57) ABSTRACT

An air purifying device (10) for reducing risk of air borne infection. The device has a hollow body having an inlet (14), an outlet (16) and an internal surface comprising a metal oxide layer. A fan (18) moves air through the body in contact with the surface, while a UV source comprises a first section for producing ozone in the air and a second section for producing hydroxyl ions. In one form the device may operate in two modes, a first mode for air purification where ozone output is minimised or not generated at all and a second mode for surface and air sterilisation where ozone output is maximised.

### TECHNICAL FIELD

The present invention relates to an air purifying/sanitiser system/device configured for conditioning air passing therethrough and removing undesirable properties thereof. The invention can also be described as a photocatalytic oxidisation processing unit and/or a biological control unit (BCU).

### BACKGROUND TO THE INVENTION

Airborne toxins, of both organic and inorganic origin, are a known problem in many environments; for example, pathogens in a hospital, clinic or nursing home. A device to reliably remove undesirable risks from the air is potentially of great value and utility, representing a clear need at all times, and particularly during pandemics.

### SUMMARY OF THE INVENTION

The invention seeks to provide an air purifying device for sanitising air; e.g. to make air safer for breathing and/or reduce the risk of an infection spreading.

According to a first aspect of the invention, an air purifying system is provided as outlined in claim 1. The device comprises: a hollow body having a longitudinal axis, including an inlet and an outlet; one or more means for moving air or a gas through the hollow body at a sufficient volumetric flow rate; a surface within the hollow body, the surface comprising a metal oxide (herein is also defined as covering metal dioxides); and at least one source of UV light within the body to direct UV light onto the surface comprising the metal oxide; wherein irradiation of the metal oxide with the UV light produces hydroxyl ions and processes the air or gas moving therethrough by effectively destroying toxins. Air that is ejected from the outlet is preferably sanitised for safer breathing and a reduced risk of a pathogen being spread.

The device may also be described as comprising a hollow body; one or more means for moving air or a gas through the hollow body; a surface within the hollow body, the surface comprising a metal oxide; and a source of UV light within the body to direct UV light onto the surface comprising the metal oxide; wherein irradiation of the metal oxide with the UV light produces hydroxyl ions and/or ozone for destroying particles in the air, e.g. associated with viruses, odours, mould, fungus spores and other toxins.

In one form the UV source is a lamp designed in two parts welded together in series or, alternatively, a continuous single type UV lamp of various shapes or multiples thereof. A first part may comprise a high ozone producing quartz (e.g. at a wavelength of 170-195, preferably 185 nm, and a second part comprises hydroxyl ion producing quartz (e.g. at a wavelength of 240-270, preferably 254 nm. Hydroxyl ions are significantly more reactive than ozone, resulting in two effects: (i) it destroys the ozone generated in the first part of the reaction chamber and (ii) works as a highly efficient oxidizer for further disinfection.

The bulb may be any length, e.g. up to 900 mm, and may be all ozone or hydroxyl generating, or any ratio of the two. It is expected that an effective ratio of ozone to hydroxyl bulb length may be 1:8 (e.g. for an 840 mm total length bulb, 90 mm ozone-producing section and a 750 mm hydroxyl producing section) or less, such as 1:10, 1:12, 1:15, 1:16, 1:20. Ozone output of the device can be measured to ensure compliance with safety regulations and, if exceeding upon normal use, a filter can be installed at the outlet end to remove excess ozone not destroyed by the hydroxyl section. In general the ratio of ozone-producing lamp length to hydroxyl-producing lamp length is determined by measuring ozone output and deciding if this is safe for use.

In one form the shape of the bulb could be “U” or “S” shaped to give a greater effective length (e.g. double or triple) of the bulb than 900 mm. A corkscrew or other shapes are also possible that increase effective length.

An all-hydroxyl-producing bulb may be used alone or in combination with an alternative ozone generating means, such as a plate-type generator arranged upstream of the hydroxyl ion producing bulb.

In one form an inner surface of the tube has been coated with a titanium oxide/dioxide and/or a zinc oxide/dioxide. A base coat may be zinc oxide/dioxide and/or including titanium dioxide. A particularly advantageous blend has been selected to be Anatase 48 g/80% and Rutile 12 g/20%. Said blend results in unexpected gains in efficiency for pathogen destruction, by increasing the number of active ions for decontamination. Rutile and Anatase have different sized particles, Anatase being a nano-particle that creates a different effect and efficiency when mixed with the Rutile.

The effective light-contacted surface area of the hollow body may be increased by introducing surface features. For example: castellations, corrugations, waves (in cross section) or the like will provide more surface area for exposure to the UV light and, hence, greater capacity to produce a reaction for disinfecting air passing through.

Surface features could be twisted/spiraled, like rifling in a barrel, to improve mixing of air within the device and overall efficiency.

The oxide/dioxide coating does not produce ozone molecules by itself. Ozone is produced, for example, by the 185 nm section of the high purity fused quartz tubing and enhanced by the free radicals (ozone) formed at the surface of the Titanium Dioxide.

The second part of the process; e.g. the 254 nm section of the quartz tube for example, produces Hydroxyl ions, which is also enhanced by the reaction between the oxide/dioxide coating of a particular blend ratio as mentioned above, and the different light wavelength being emitted by the 254 nm quartz.

According to a further aspect, the present invention also provides a method for purifying air comprising use of a device as described herein. The method may produce nitric oxide catalytically from air or a gas by irradiating a metal oxide with UV light. Nitric oxide has a biocidal effect.

Irradiation of the metal oxide/dioxide with the UV light may produce nitric oxide from the air or the gas thereby modifying the air or gas released by the device. The device may produce an output concentration of nitric oxide of, for example, greater than 0.25 ppm or up to or more than 2.0 ppm. Other amounts are contemplated. In certain embodiments, the device produces an output concentration of nitric oxide of in the range from 0.5 to 2.0 ppm, 1.0 to 2.0 ppm. 1.5 to 2.0 ppm, 1.0 to 2.00 ppm or 1.5 to 2.0 ppm. Other ranges are contemplated.

The device may operate in a controlled atmosphere setting. In certain embodiments, the controlled atmosphere setting comprises a surgical theatre, cool room, cold room, storage room, elevator/lift, gymnasium, health club or a vehicle used for transport (e.g. a train, car, boat or aeroplane). In certain embodiments, the device may operate in a storage container or a shipping container. Other types of closed settings or systems are contemplated (e.g. public spaces, waiting rooms, offices, domestic dwellings, etc.).

As mentioned above, an enclosed space of particular consideration is an elevator (e.g. in a hospital) where air within can be purified during use or, in an ozone flooding mode, during periodic non-use when the doors are closed. When the doors open, ozone-rich air is quickly dissipated.

A particular feature of the invention is to provide a high-ozone output mode to flood a space with ozone for the purpose of destroying airborne (and surface resident) toxins. The ozone may be left to naturally dissipate or the ozone-rich air can be drawn through the device with hydroxyl-producing bulb active to remove ozone more quickly than natural dissipation allows.

The device is expected to operate in a setting held at ambient temperature, e.g. 25° C. or less, 20° C. or less, 15° C. or less, 10° C. or less, 4° C. or less, or 2° C. or less. Other temperatures are contemplated, such as greater than 25° C.

The device preferably operates in a normal oxygen environment or atmosphere; i.e. around 21% oxygen. In certain embodiments, the device may operate in a low oxygen environment or atmosphere. In certain embodiments, the device operates in an atmosphere comprising 5% oxygen or less. In certain embodiments, the device operates in an atmosphere comprising 2% oxygen or less.

In certain embodiments, the device is operable continuously. However, the device may operate intermittently. The device may operate for 1 hour or greater, 6 hours or greater, 12 hours or greater, 18 hours or greater, 1 day or greater, 2 days or greater, or 1 week or greater.

The device operates to reduce volatile organic compounds in the air or the surrounding gas, thereby modifying the air or gas released by the device for improved health benefits.

The device preferably reduces the concentration of volatile organic compounds in air passing therethrough to 25 ppm or less, or 20 ppm or less, or 0.2 ppm or less, or 0.05 ppm or less, or less than the level of detection using current measuring technology, or at least less than safety regulations dictate. The device also operates to reduce the concentration of microorganisms in the air or the gas passing through.

In certain embodiments, the hollow body comprises a substantially cylindrical tube. Other configurations are contemplated, including tubes having a rectangular, square, pentagon, hexagon, other polygonal shapes or oval cross-section. Many individual units may be implemented together as required, i.e. combined together for any given environment and the efficacy required.

The invention is intended to be scalable for different applications of use and capacity.

In certain embodiments, the one or more means for moving air or a gas through the hollow body comprises one or more fans. Other means are contemplated, such as a source of air or a gas held under pressure and directed into the device. The fan may push or draw air or gas through the device; e.g. a fan at one end of the hollow tube to push or draw air or gas through the tube. In certain embodiments, the fan comprises a power of 10 to 50 W or greater, however, other power outputs are contemplated. In certain embodiments, the one or more means for moving air or a gas through the hollow body produces an air or gas flow between 10 and 1000 cubic meters per hour depending on the application. Other flow rates, both higher and lower, are contemplated for various specialized applications (e.g. personal protection equipment (PPE, anti-bacterial face masks), continuous positive airways pressure (CPAP) machines where low volume/airflow is required, to large HVAC systems, e.g. in airports and large public spaces, where very high volume and airflow is required. The one or more means for moving air or a gas through the hollow body may produce an air or gas flow of 10 m³ per hour or greater, 25 m³ per hour or greater, 50 m³ per hour or greater, 75 m³ per hour or greater, or 100 m³ per hour or greater, or 125 m³ per hour or greater, or 150 m³ per hour or greater. It is also contemplated to use a remote fan (not internally fitted within the device) to move the required volume of air or gas through the device e.g. a fan provided by an existing HVAC system. The system may be modular such that a fan unit can be coupled to one or more UV tube sections in series, e.g. an ozone tube, an hydroxyl tube, a fan unit at one or both ends or therebetween, and a filter unit at any stage. Sections can be interchangeable and assembled according to best practice instructions. Such a system enables damaged components to be swapped for continued use of the operational device.

The fan(s) speed can be adjusted to control the airflow passing through the device, thereby optimising the volume and dwell time for air/gas passing through the device for operating efficiency in the given environment. The fan(s) and light source(s) can be manually adjusted or switched on/off or automated depending on personal preference or by monitoring of the air quality in the environment through sensors. The adjustments can be made manually or via an application (e.g. smartphone, tablet or personal computer) using WiFi®, Bluetooth®, hard-wired or any other method available to achieve the required efficacy. Further automation or preset values may be considered through an algorithm or by using a third-party proprietary air monitoring system to control the device.

As mentioned, the surface within the body (which may be directly on the body and/or on an intermediate or inserted surface) comprises a metal oxide/dioxide coating and/or a surface embedded with a metal oxide(s) or dioxides.

In certain embodiments, the method of coating comprises dipping the surface to be coated in a solution of the selected metal oxide(s) blend. In certain embodiments, the method of coating comprises painting and/or spraying the surface to be coated with a solution of the metal oxide(s). In certain embodiments, the method of coating comprises dipping the surface to be coated in a solution of the metal oxide(s) and drying the surface. In certain embodiments, the method of coating comprises painting and/or spraying the surface to be coated with a solution of the metal oxide(s) and drying the surface. In certain embodiments, the surface comprises one or more coatings.

In certain embodiments, the metal oxide/dioxide comprises one or more of a zinc oxide and a titanium oxide/dioxide

The metal oxide comprises a titanium oxide/dioxide. In certain examples, the metal oxide comprises a rutile titanium oxide/dioxide. In certain examples, the titanium dioxide comprises an anatase titanium oxide. However, preferably, the metal oxide comprises a rutile titanium oxide and an anatase titanium oxide or dioxide blend of 20/80 by weight. In certain embodiments, the metal oxide or dioxides additionally comprises a zinc oxide with the titanium oxide.

In certain embodiments, the surface comprising a metal oxide comprises one or more undercoats of a metal oxide(s). In certain embodiments, the surface comprising a metal oxide comprises one or more undercoats of a metal oxide(s) and one or more overcoats of a metal oxide(s).

In certain embodiments, the undercoat comprises an undercoat comprising a zinc oxide. In certain embodiments, the undercoat comprises an undercoat of a manganese oxide. In certain embodiments, the undercoat comprises an undercoat of a zinc oxide and/or a manganese oxide and/or a cerium oxide. In certain embodiments, the metal oxide comprises an undercoat of a zinc oxide and an overcoat of a rutile titanium oxide, an anatase titanium oxide and a zinc oxide.

In certain embodiments, the hollow body comprises a metal and/or a metal alloy. In certain embodiments, the hollow body is composed of a metal and/or a metal alloy. Other types of materials are contemplated, for example a glass. In certain embodiments, the hollow body is a tube and the tube is composed of a metal and/or a metal alloy. In certain embodiments, the hollow body is composed of a steel, such as a stainless steel. In certain embodiments, the hollow body is a tube and the tube is fabricated from sheet materials, e.g. composed of a steel, such as a stainless steel.

In certain embodiments, the surface within the hollow body comprising a metal oxide comprises an inner surface of the hollow body. For example, all or part of the inside surface of a tube may be coated with the metal oxide(s).

In certain embodiments, the surface within the hollow body comprises a metal oxide comprising an insert (for example a sleeve) which can be placed inside the hollow body. In certain embodiments, the surface within the hollow body comprising a metal oxide comprises one or more plates placed inside the hollow body (e.g. diffusers that inhibit light being visible from outside the device which may harm a user). In certain embodiments, the surface within the hollow body comprising a metal oxide comprises all or part of the inner surface of the hollow body. In certain embodiments, the surface within the hollow body comprising a metal oxide comprises substantially the entire inner surface of the hollow body.

In certain embodiments, the surface within the tube comprising a metal oxide comprises substantially the entire inner/inside surface of the tube. In certain embodiments, the surface within the hollow body comprising a metal oxide is scored and/or treated prior to coating with the metal oxide.

In certain embodiments, the surface within the hollow body comprising a metal oxide is scored prior to coating with the metal oxide/dioxide; e.g. by chemically and/or mechanically scoring or etching prior to coating with the metal oxide. In certain embodiments, the surface comprising a metal oxide is scored or treated with a solution of an acid and/or a salt and/or a detergent. Examples of salts include ferric chloride or copper sulphate. Examples of acids include hydrochloric acid, nitric acid, or sulphuric acid, which can be used to chemically treat the surface to score or etch the surface.

In certain embodiments, the solution for scoring comprises a concentration of ferric salt of 10 to 25% (w/w). In certain embodiments, the solution for scoring comprises a concentration of ferric salt of 15 to 25% (w/w), 20 to 25% (w/w), 10 to 20%) (w/w), 15 to 20% (w/w), or 10 to 15% (w/w). Other amounts are contemplated. In certain embodiments, the solution comprises a concentration of detergent of 1 to 5% (w/w), 2 to 5% (w/w), 3 to 5% (w/w), 4 to 5% (w/w), 1 to 4% (w/w), 2 to 4% (w/w), 4 to 4% (w/w), 1 to 3% (w/w), 2 to 3% (w/w) or 1 to 2% (w/w). Other amounts are contemplated.

In certain embodiments, the detergent comprises an anionic detergent. In certain embodiments, the detergent comprises sodium laurel sulphate and/or a derivative thereof. Other types of detergents are contemplated.

In certain embodiments, the source of UV light comprises a UV-C light source. In certain embodiments, the source of UV light emits light in a range from 100 to 290 nm. In certain embodiments, the source of UV light comprises a UV-C lamp or tube. In certain embodiments, the source of UV light has a power output of 8 W or greater, 10 W or greater, 20 W or greater, 30 W or greater, 40 W or greater, 50 W or greater, 60 W or greater, or 70 W or greater.

In certain embodiments, the device, at the outlet, produces substantially no ozone.

While the invention is primarily developed for the purpose of purifying air in health care and analogous environments, the technology is also applicable to domestic/Industrial applications; filter/purification systems for domestic HVAC systems, i.e. office buildings and public gathering places; potentially scaled, up or down, for the required environment; cleaning and raising the air quality within the beauty Industry, e.g. anti-ageing; removing allergies from the air in all environments—hay fever, asthma and other respiratory issues; air quality and safety for applications like underground rail systems; manufacturing “clean rooms” where a clean air policy is necessary; automotive applications including motor sport where the air quality in a closed cockpit cars can be extremely poor and/or incorporated into a semi-sealed crash helmet (fireproof) that is required by regulations; military purposes such as advanced gas mask requirements (chemical and biological warfare), support vehicles, submarines, naval vessels and requirements, aircraft of all kinds; public and personal transport applications such as buses, trains, aeroplanes, boats.

Particular medical applications include hospital isolation wards where the removal of viruses is necessary for patient and staff wellbeing; nursing homes; doctor and dental surgeries; laboratories of all kinds; integrated into personal protection equipment (PPE)—face masks, e.g. by the use of miniaturised LEDs.

In connection with the above multiple uses and installations further modifications and features may be incorporated. For example: the outer tube/casing can be made of many material types such as a carbon kevlar mix, stainless steel, aluminium, plastics, composites; the outer tube diameter and length can be varied and scaled up or down for different applications and different airflow volume requirements; the fan size can be varied to be matched to the specific air device dependent on power supply, mass of air to be circulated and length/diameter of the system; the inlet and outlet of the device can be varied in length, diameter, shape and number of inlets and outlets to account for unique installations and clean air distribution requirements.

Devices according to the invention may be fitted with an air filter system (e.g. a high-efficiency particulate air—HEPA filter or a carbon or active carbon filter) at or close to the inlet and/or outlet of the system, to prevent dust and other particulates from entering the device and contaminating the (TiO₂) lining and/or from subsequently exiting the device. Without a pre-filter, the coating may become compromised and efficiency of the device may degrade in particularly dirty environments. However, it is noteworthy that for some HVAC applications a fan and/or filter arrangement may not be necessary due to integration into an existing HVAC system. One or more air filters (e.g. HEPA) can be incorporated at either or both ends of the tube. Inclusion of a filter has considerable benefits to clean room application and environments where a particulate free (nano-dust particles) atmosphere is required. A downstream filter may be incorporated to remove any excess ozone, i.e. to reduce emissions to regulated levels. Such filters may be monitored for end-of-life indications.

Preferably the UV source entails two sections, defining two zones of the device, i.e. an ozone producing zone that destroys contaminants in the air passing therethrough and hydroxyl ion producing zone which further enhances disinfection and destroys the ozone generated in the upstream portion before it can be vented to atmosphere. Either the ozone or hydroxyl generating lengths of the bulb can be varied as can the ratio between the two. A full hydroxyl only bulb may be suitable in some applications, whereas an ozone only bulb may be used in other applications.

The unit may be mounted by many different methods, e.g. floor mounted, wall mounted, hung from ceiling, free standing, at an angle, packaged as part of a domestic, industrial, or automotive etc. HVAC system.

The operating power supply may be variable and configured for different applications, e.g. 12V to 48V or greater for current automotive requirements and/or battery powered vehicles (EVs or hybrids). Various battery types and voltages may be possible for portable, PPE applications—face masks etc. The required voltage for the system can be achieved by the addition of a voltage regulator or transformer as required. Any voltage to suit EVs or hybrid vehicles depending on the voltage architecture of the vehicle. Any grid electricity supply e.g. 110V; 220/240V; 3 phase supplies etc.

### BRIEF DESCRIPTION OF THE DRAWINGS

FIGS. 1 and 2 show an external perspective view, from two different angles, of an air purification device according to one embodiment;

FIG. 3 shows a cross-section side view of the inside of a device according to one embodiment;

FIG. 4 shows an exploded view of the components of an air modifying device according to one embodiment;

FIG. 5 shows a schematic view of a UV double element light source, suitable for incorporation with the device;

FIG. 6 shows a larger diameter format air purification device, according to a second embodiment;

FIG. 7 shows a cross-section side view of the inside of the device according to the second embodiment;

FIGS. 8 and 9 show exploded views (underneath and overviews) of components of the device according to the second embodiment, divided into four compartments for a four-bulb system;

FIG. 10 shows a perspective external view of a device according to the invention, suited for HVAC use;

FIG. 11 shows an exploded view of components from the device according to FIG. 10; and

FIG. 12 shows a perspective view of a cartridge for a plate-type ozone generator and ballast for fitting to an inlet of a tube, e.g. as shown in FIGS. 1 to 4.

### DETAILED DESCRIPTION OF THE INVENTION

The embodiments described herein relate to an air purifying device, alternately referred to as a sanitiser or the like. It should be appreciated that embodiments of the present invention and the descriptions and figures herein are not limited to specifics. The embodiments described herein may have a variety of different applications including, for example, cleaning of air for any applicable use where the health of human beings and animals is the primary consideration.

The term oxide also refers to dioxide and vice versa.

The following description presents exemplary embodiments and, together with the drawings, serves to explain principles of the invention. The scope of the invention is not intended, however, to be limited to the precise details of the embodiments, since variations will be apparent to a skilled person and are deemed also to be covered by the description. Terms for components used herein should be given a broad interpretation that also encompasses equivalent functions and features. In some cases, several alternative terms (synonyms) for structural features have been provided but such terms are not intended to be exhaustive.

Descriptive terms should also be given the broadest possible interpretation; e.g. the term “comprising” as used in this specification means “consisting at least in part of” such that interpreting each statement in this specification that includes the term “comprising”, features other than that or those prefaced by the term may also be present. Related terms such as “comprise” and “comprises” are to be interpreted in the same manner. Directional terms such as “vertical”, “horizontal”, “up”, “down”, “top”, “bottom”, “upper” and “lower” or similar are used for convenience of explanation usually with reference to the illustrations and are not intended to be ultimately limiting if an equivalent function can be achieved with an alternative dimension and/or direction.

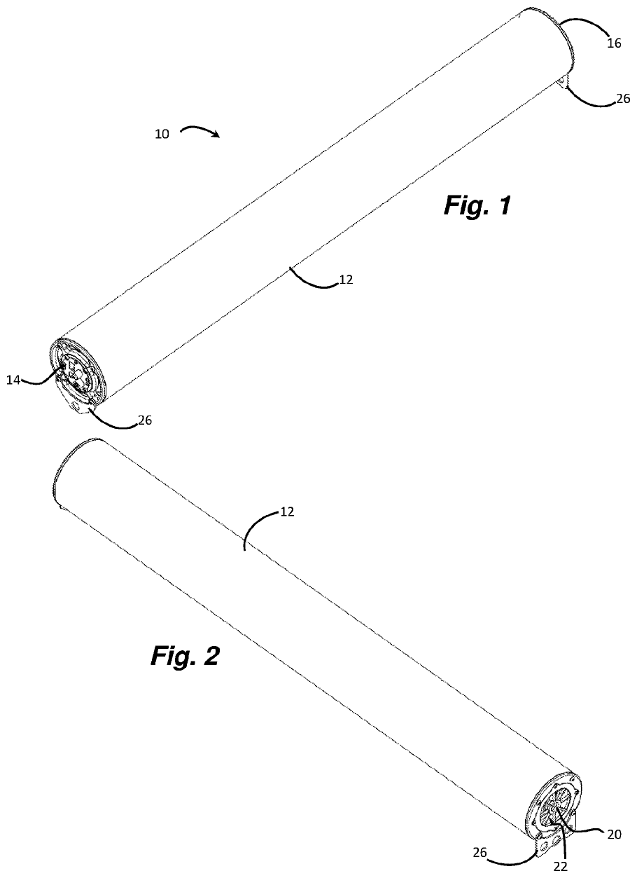

The present disclosure relates to an air modifying device for purifying air to improve health conditions for humans and animals. FIG. 1 shows an external view of an air modifying device 10 according to one embodiment. The device 10 comprises a cylindrical stainless steel (e.g. UNS S30400—grade 304) tube 12, which may have a length of 100-300 cm (e.g. 1.26 m), an outside diameter of 10-30 cm (e.g. 15 cm) and a wall thickness of 1 to 5 mm.

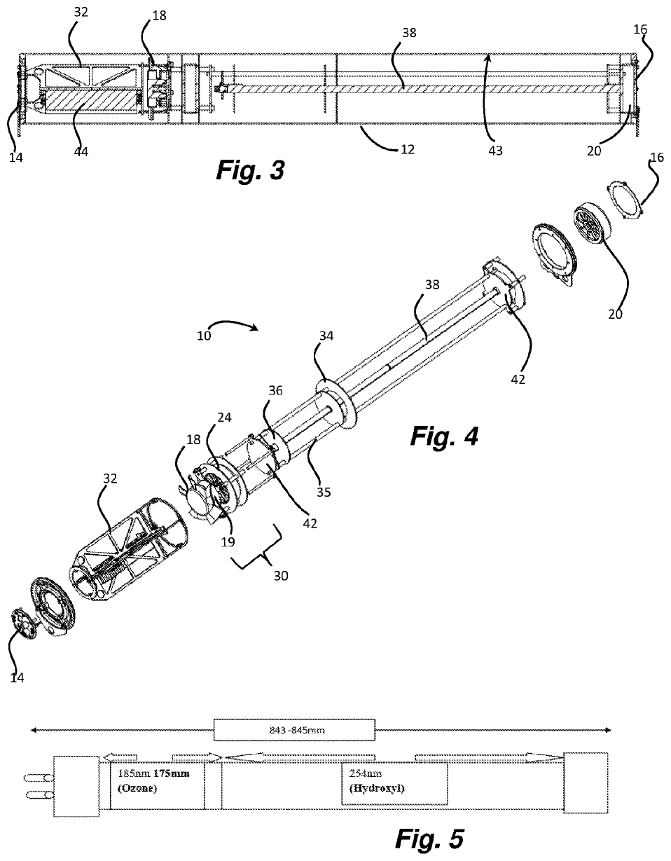

The tube 12 of the air modifying device 10 comprises two open ends, inlet 14 and outlet 16, which permit air or other gas to move therethrough the tube 12. Inside the tube 12 is a surface coated with a catalytic metal oxide(s) and a UVC lamp. Referring to FIGS. 3 and 4, a fan unit 18 is located at one end of the device (in the illustrated embodiment, proximate inlet 14), which draws air into the device from the atmosphere and moves the air through the device 10, so that the air is expelled at the outlet end 16 of the device. Typically, the fan unit 18 comprises an electrically powered fan of any suitable power, for example approximately 20 W to 50 W and it will be appreciated than the fan can be fitted within or exterior to the tube or fabricated body 12.

At the outlet end of the tube 16 is an optional vent/air restrictor or filter 20 sleeved into the inside of the tube 12, which assists in circulating the air expelled from the device 10 through the optional vents 22. The filter 20 may, for example, have a composition for removing ozone from air passing therethrough.

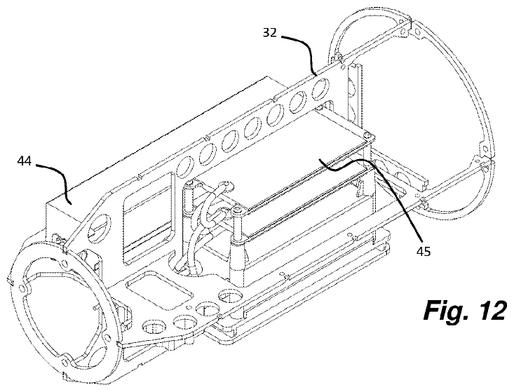

The device may be powered by either a 110V power source or a 220V/240V power source, or any other applicable specification, e.g. for portable use. The electrical components may be housed externally, e.g. under a vented cover, to assist with cooling the electrical components therein, or internally within tube 12; e.g. at a first section proximate the inlet 14. Lamp ballast 44 is shown as part of a cartridge 32 (FIG. 12) that may be inserted into tube 12. The device may also have an electronic light on the outside of the device indicating that the device is on/off and/or the UV light within the device is operating. The device may be remotely controlled, and/or be part of an integrated controlled system. Generally, the device may have variable speed settings for the fan and be operated via WiFi®, Bluetooth®, and/or via a remote control or application. An air quality monitor may also be integrated or separate to the device to control the efficiency of the device either manually or automatically.

The device 10 also includes one or several means for supporting the device in a vertical, angled and/or horizontal position, such as suitable brackets 26 or hooks appropriately extending from the device and which permit hanging or attachment of the device to a wall or ceiling or other object, although it will be appreciated that the device 10 may also be adapted for free-standing portable use.

The device is scalable and may also be produced, and which may be used, for example, as a free-standing unit on legs. Such a device may be used in smaller areas or to supplement the activity of one or more larger units. A smaller device typically may have an overall length of around 50 cm and a tube diameter of 10 cm.

Referring to FIGS. 3 and 4, an internal view of the device 10 is shown according to one embodiment. As mentioned the illustrated fan unit 18 is located near the inlet end 14 (although may be located at the other end, or with a fan at both ends, mid-way or externally) to draw air through the tube 12 and uses an electric motor 19. The fan unit 18 is part of a fan assembly 30, which at one end includes a component 24 that slides into the inside of the tube 12 and is secured in place. The fan assembly 30 may be located either before or after a first section of the tube housing electronic components 44. A pre-filter may be included to remove particulates before they enter the inside of the tube 12. Provision of an air filter protects internal components and especially the sensitive coating of the internal walls.

Inside the tube 12 is located an ultraviolet (UV) lamp 38 (shown in more detail by FIG. 5), which is typically a dual quartz 70 W ultraviolet (UV) lamp and which emits in the UVC range. The UV lamp 38 is operated by an electrical junction and suitable lamp ballast 44, which are positioned under a cover or inside the unit upon cartridge 32. The UV lamp can be supported by brackets 42 (which may also act as diffusers to prevent UV lamp 38 being directly visible from an external position), using clamps and screws to support the UV lamp 38 positioned at the centre of the tube 12. If more than one bulb is fitted, these can be fitted off the centre line of the tube. The lamp(s) can be retained in a fashion, e.g. in a removeable module, that allows easy access for replacement and servicing.

FIGS. 3 and 4 show a partition plate or plates 34 located, mid-way over lamp 38, upon support rods 35. Plate 34 forms a wall to prevent light from a first (ozone-producing) section of lamp 38 from contacting an inner coated wall surface of a second (hydroxyl-producing) section of lamp. Ozone-rich air may pass through a gap between the plates 34 where it is subjected to hydroxyl ions in the second section.

The lamp may include a diffuser plate 36 at one or both ends for preventing direct visual access to lamp 38.

By way of example, the device 10 may utilise an AC (240V-50 Hz or 110V-60 Hz) supply cable with interior wiring to the UV lamp 38 and ballast 44 and the fan unit 18. The device 10 may also include a power monitoring accessory to remotely sense failure of the fan or lamp, and/or an electronic light on the device indicating that the device is on and the UV light within the device is operating.

In the embodiment shown, a catalytic coating 43 is applied to the inside surface of the tube 12 so that UV irradiation is directed onto that surface.

However, it will be appreciated that the catalytic coating may only be applied to a portion of the inside surface of the tube 12, or in another embodiment, one or more plates coated with the catalytic coating may be mounted inside the device 12. In another embodiment, replaceable catalyst coated inserts can be used, and can be comprised of stainless steel, silica, or a UV-resistant substrates on which the catalysts are imbedded.

In the embodiment shown, a catalytic coating 43 is applied to the inside of an aluminium tube 12.

In the embodiment shown and by way of preferred example, the coating may be applied by first treating the internal surfaces of the stainless steel tube 12 with Ferric Chloride. Following this treatment, the tube is washed with water and allowed to dry. A wet coating comprised of a mixture of 20% Rutile titanium oxide (e.g. CAS NO.: 13463-67-7) and 80% Anatase titanium oxide (e.g. CAS NO.: 12065-65-5) by weight is applied as a mixed solution by a pressed roller method to scored internal surfaces to produce the final coating. It will be appreciated that other oxides, and mixture of oxides, may be utilised. For example, the oxides may comprise a mixture of rutile titanium, anatase titanium oxide (still maintaining a 20/80 relative mix of rutile/anatase) and zinc oxide.

In the embodiment shown, the device 10 utilises a high output UV lamp 38 housed centrally in the stainless steel tube 12, that has been selectively coated internally with metal oxide catalysts to ablate volatile organic compounds (VOCs) to CO₂ and water, and also generate and maintain desirable concentrations of nitric oxide (NO) for disinfectant purposes. More than one bulb may be fitted about a centre line for various applications. Indeed, bulbs of different ozone to hydroxyl length ratios and shapes can be used in the same device to give the desired efficiency.

The catalytic coating, when acted on by UV light, provides a number of advantages: (i) producing hydroxyl radicals (OH); (ii) ablating Volatile Organic Compounds (VOC's); and (iii) the device acts as a biocide, to significantly disinfect and deodorise air passing therethrough.

Referring to FIG. 4, an exploded view of the device 10 is shown. Again, at one end 14 of the device 10 a fan assembly 30 is shown, which includes fan 18 (driven by fan motor 19), and a frame 32 that may support electrical components 44 and any supplementary functional features e.g. the ballast for the bulbs and/or an ozone generator as pictured by FIG. 12.

The device 10 may also include an air filter either before or after the fan unit 18, to remove particulates before they enter the interior of tube 12 proximate bulb 38. At the other end of the tube 16 is a air diffuser/filter 20, having front and rear faces. The diffuser/filter 20 assists with circulation of the modified air as it leaves the device 10 and can control the dwell time for airflow passing through the tube 12. A filter may, in principle, be installed mid-way in the tube between the ozone and hydroxyl sections. In such an arrangement, the filter may be side-loaded into a slot for replacement, or part of a modular system where components are connected in series.

Inside the tube 12 is located an ultraviolet (UVC) lamp 38. The UV lamp 38 and other components are operated by an electrical junction and a suitable lamp ballast 44, e.g. supported by an electronic ballast tray 32.

The UV lamp 38 is supported by a frame comprised of support brackets 42, rods 35 and any optional intermediate partitions 34, so as to position the UV lamp 38 at the centre of the tube 12, where UV light from the lamp can irradiate the inner surface 43 of the tube coated with metal oxide(s) 12 to produce ozone and hydroxyl ions. If more than one bulb is fitted, these may be mounted off the centre line of the tube.

FIG. 5 illustrates a schematic example of internal UV lamp 38, comprised of quartz sections joined/welded together in series. The first part extends, by way of example, 175 mm capable of producing light of 185 nm in wavelength, from a high-ozone producing quartz. The second part extends, by way of example, 668 mm capable of producing light of 254 nm. The second section produces Hydroxyl ions. Each section creates different oxidizers which have dissimilar, complementary disinfectant properties. In practice the ozone-producing length may be shorter than pictured, e.g. 44 mm, still with a total length of 845 mm. In this way, the ratio of first to second section is expected to be less than 1:4, possibly approximately 1:18. Alternatively, the bulb may have no ozone-producing section at all. A hydroxyl-only bulb provides disinfectant properties on its own and may be used in combination with an alternative upstream ozone-generating source such as a plate-type generator (see FIG. 12) or a separate ozone generator unit.

Nevertheless, as illustrated in FIG. 5, the sections of the UV source effectively partition the device 10 into at least two zones. Within the first zone a high concentration of ozone is generated from reaction with oxygen in the atmospheric air drawn into the device at the inlet. The ozone destroys contaminants in that air. Within the second zone hydroxyl ions are generated providing a further enhanced disinfection process and converting the ozone back to oxygen for safe release back to atmosphere. A filter at or near the exit of the device can be fitted to remove any remaining unwanted gases produced by the device 20 or proximate the location of partition 34. An external filter can also be implemented if required. Filters should be removeable and replaceable when end-of-life is reached. An end of life service indicator (ELSI) may be implemented for filters.

The exemplary embodiment of UV source is a lamp. However further embodiments may incorporate LEDs configured as a UV source, either instead of or complimentary to a lamp. For example, a first zone and second zone of the device may each implement a different or combination of UV sources. As previously stated, the hydroxyl-generating section is considered the most necessary zone since it could be used to enhance ozone dissipation in a room flooded with ozone by an ozone generating means.

An example of lamp embodiment is outlined below. Preference is given to a high output design, however, that requires greater time to attain maximum operational output:

1. A UVC lamp, e.g. a G36T15N, which utilises liquid mercury and is driven at ^{˜}430 mA to achieve a lamp wall temperature of around 450° C. to achieve optimum mercury vapour pressure and maximise UVC output.

2. A high output UVC lamp, e.g. a GHO36T15N, utilising liquid mercury and driven at ^{˜}800 to 1000 mA to achieve a lamp wall temperature of around 850° C. to achieve an optimum mercury vapour pressure for maximising UVC output. A GHO lamps use ‘Cold Spot’ technology as the Lamp is designed to create a cold spot behind the filaments which will be around 450° C. while the rest of the Lamp will be at ^{˜}850° C. The coldest spot within the lamp determines the mercury vapour pressure.

In connection with use of the device, as described herein, in certain embodiments the device can be described as a photo-catalytic unit utilising a high output ultraviolet (UV) lamp housed in a purpose built tube that has been selectively coated internally with catalysts that ablate VOCs to CO₂ and water.

In one form the invention can be configured as an ozone and/or hydroxyl ion generator. Particularly, through the enhancement or addition of one or more ozone generating UV lamp(s) in the tube, e.g. that can be varied in length and number. The electronic ballast/controller may be configured to act as a switch and/or timer for switching on/off any or all the individual UV light sources to create the amount and timing of ozone generation required, for the purpose of generating larger amounts of ozone when a space is not occupied by a person or animal.

Such a function may be primarily used for treating of surfaces within an environment; i.e. by ozone flooding. The ozone can be produced by the reaction of the UV lamp(s) with the coating or through a plate type ozone generator incorporated as a module within the device, e.g. as illustrated by FIG. 12, or separate to the device. Either method can be controlled manually or through the automated algorithmic system.

A plate-type ozone generator 45 is illustrated by FIG. 12, which is a component that may be incorporated with frame 32, along with lamp ballast 44, at an inlet 14 of the device shown in FIGS. 1 to 4. As mentioned, the plate-type ozone generator can be operated together or separately to the UV light sources and the operation can be timed for switching on and off. A modular device of multiple sections may be connected together in series and powered by a common energy source.

In an ozone generating mode, the Hydroxyl ion generating portion of a double filament UV lamp (as described above) can be turned off so as not to inhibit the quantity of ozone generated and expelled through the exit of the device, or separately if a remote ozone generator is used. If a filter is used at the exit of the device, a bypass arrangement can be incorporated to release the un-filtered ozone into the environment. For example, fan 18 can be reversed to make inlet 14 an outlet. In this embodiment, air flow within the device is reversible depending upon which mode is selected.

The large/enhanced amount of ozone emitted from the purifying device will be able to flood a given environment to effectively clean and sanitise surfaces during periods (e.g. overnight or weekends) when personnel are not present. In this way the invention can be configured as a single device to remove air borne VOC's/viruses and is also an efficient ozone generator to sterilise surfaces. The amount and concentration of ozone flooding can be monitored and automatically controlled by the remote or internal monitoring system. In some embodiments a sensor will be mounted externally within the closed environment.

Ozone flooded into a volume will typically dissipate to safe levels within a predetermined time (e.g. a few hours), leaving surfaces sterilised. Ozone dissipation can be enhanced by operating the device in a hydroxyl-only mode, where ozone-rich air drawn through the device is processed to remove ozone. When personnel return, the configuration mode of the device may be switched to air purification in accordance with the substantive description above, which is safe in the presence of humans and animals. Warning lights and/or a software application can notify personnel when the environment is safe to re-enter.

The invention can be broadly described as an air purifying device for reducing risk of air borne infection. The device has a hollow body having an inlet (14), an outlet (16) and an internal surface comprising an active layer. A fan or fans (18) move air through the body in contact with the surface, while a first section (e.g. of a UV source) produces ozone in the air and a second section (e.g. of a UV source) produces hydroxyl ions. The ratio of one to the other can be varied by the design or dynamically during use when used in conjunction with the plate type ozone generator. In one form the device may operate in two modes, a first mode for air purification where ozone output is minimised and a second mode for surface and air sterilisation where ozone output is maximised, preferably with appropriate safety warnings.

FIG. 12 shows a plate-type ozone generator that can be operated together or separately to the UV light source(s) and the operation can be timed for switching on and off.

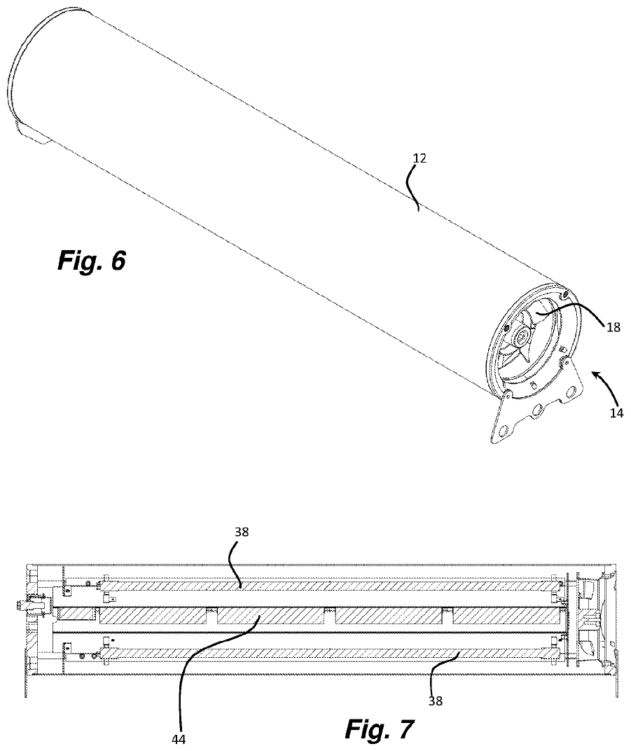

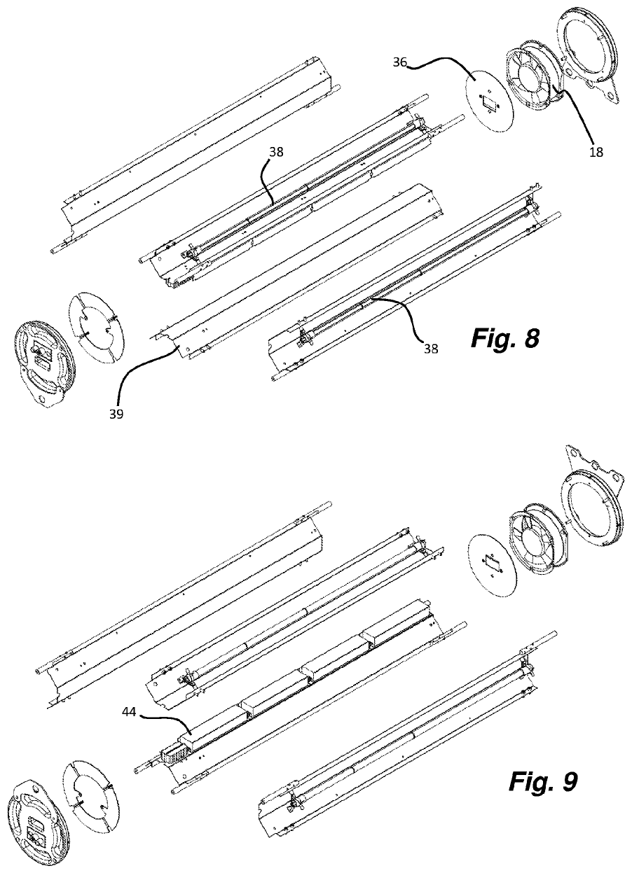

FIGS. 6 to 9 illustrate an embodiment with a larger diameter tube 12 for housing a UV source in multiple compartments. In this form, a fan 18 at the inlet 14 draws air from an external environment into the device where it passes through compartments defined by partition walls 39, each housing a UV source 38, e.g. of the type schematically shown by FIG. 5. Fans can be fitted at either or both ends, as can filters if required. Ballast electronic 44 modules provide control and power functions for the UV source and fan etc.

Each of the compartments is coated with metal dioxide as in previous embodiments. For example, FIGS. 8 and 9 show a four bulb machine with a fan on the inlet of the tube and four ballasts 44 for the bulbs mounted internally down the centre line of the tube. A diffuser 36 provides a wall preventing direct visual access by a user of the lamps 38.

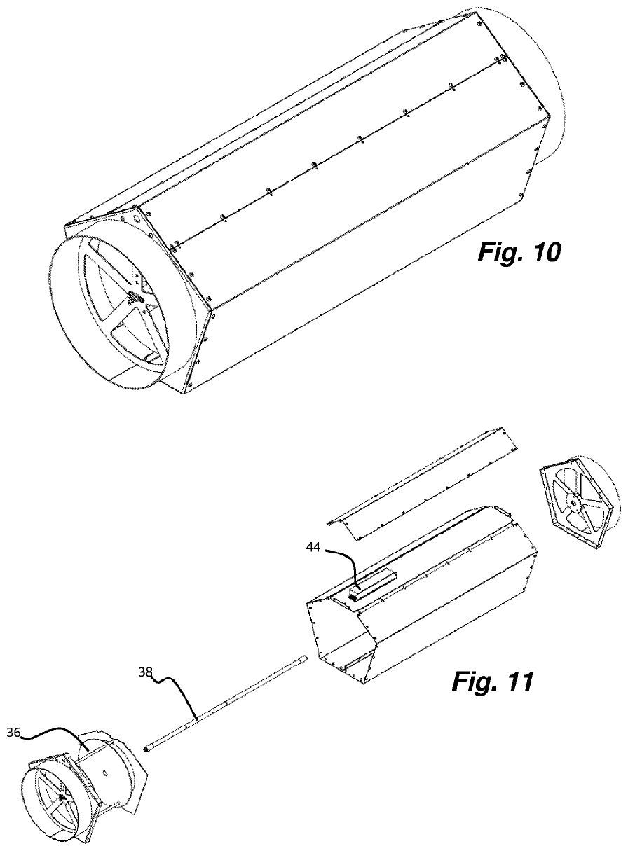

FIGS. 10 and 11 illustrate an embodiment fabricated for an HVAC system, e.g. inline with an air conditioning conduit or the like. As such, no fan is needed because airflow is already present. However, a fan may be fitted to create air flow if required. FIG. 11 shows one UV bulb and ballast, but these may be increased depending upon the application. The ballast 44 for the bulb(s) is fitted under a separate cover on the external side of the fabrication. Separate or integrated air filter(s) can be fitted at either the inlet and/or outlet as required. Mounting brackets can be fitted for different installations. The size and shape of the body can be varied to account for the number of bulbs and ballast fitted and if internal fans and filtration is required, e.g. if fitted to a lift/elevator application.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

All methods described herein can be performed in any suitable order unless indicated otherwise herein or clearly contradicted by context. The use of any and all examples, or exemplary language (e.g. “such as”) provided herein, is intended merely to better illuminate the example embodiments and does not pose a limitation on the scope of the claimed invention unless otherwise claimed. No language in the specification should be construed as indicating any non-claimed element as essential.

The description presents exemplary embodiments and, together with the drawings, serves to explain principles of the invention. The scope of the invention, however, is not intended to be limited to the precise details of the embodiments, since variations will be apparent to a skilled person and are deemed also to be covered by the description. Terms for components used herein should be given a broad interpretation that also encompasses equivalent functions and features. In some cases several alternative terms (synonyms) for structural features have been provided but such terms are not intended to be exhaustive.

1. An air purifying system for reducing risk of air borne infection, the system comprising:
a hollow body having an inlet and an outlet;
an air moving means for facilitating movement of air through the hollow body, between the inlet and outlet, such that the air is at least partly in contact with
a metal oxide or dioxide located in the body;
at least one UV source mounted within the hollow body;
wherein the at least one UV source comprises a section for producing hydroxyl ions; and
wherein the system further includes provision of a section for producing ozone, in series with and upstream of the section for producing hydroxyl ions.

2. The system according to claim 1, wherein the section for producing ozone emits a wavelength of 170-200 nm, preferably substantially 18 Snm, and the section for producing hydroxyl ions comprises a UV source that emits a wavelength of 240-270 nm, preferably substantially 254 nm.

3. The system according to claim 2, wherein the ratio of the section for producing ozone to the section for producing hydroxyl ions, in relative lengths of the sections, is less than 1:8, preferably 1:18.

4. The system according to claim 1, wherein the at least one UV source is a lamp and/or an LED.

5. The system according to claim 1, wherein the metal oxide comprises one or more of a zinc oxide, and a titanium oxide/dioxide.

6. The system according to claim 1, wherein the metal oxide comprises titanium dioxide in a mix of Anatase 80% and Rutile 20%.

7. The system according to claim 5, wherein the metal oxide comprises a zinc oxide and titanium dioxide.

8. The system according to claim 1, further comprising a filter at the inlet and/or outlet.

9. The system according to claim 1, wherein the section for producing ozone at least partially comprises a plate ozone generator.

10. The system according to claim 1, wherein the sections are modular for connection in series with an air moving section and/or a filter section and/or within an HVAC system.

11. The system according to claim 1, configured for operation in:
a first mode where ozone output from the outlet is minimised; and
a second mode where ozone output from the outlet is maximised, for the purposes of surface sterilisation.

12. The system of claim 11, comprising a switch and/or timer for switching between modes.

13. The system according to claim 11, wherein, in the second mode, the section for producing hydroxyl ions is disabled.

14. The system according to claim 1, wherein the air moving means for facilitating movement of air through the hollow body is reversible.

15. The system according to claim 1, comprising a plurality of UV sources for producing hydroxyl ions.

16. The system according to claim 1, including a means of closed loop monitoring of the external environment to control the device manually or automatically.

17. The system according to claim 1, wherein the hollow body has an internal surface with a profile to increase effective surface area exposed to the UV source.

18. Use of the system according to claim 1, for reducing the concentration of volatile organic compounds in the air, and/or reducing the concentration of viable organisms and/or spores in the air.

19. A method for improving air quality and reducing the risk of infection to a human being in an enclosed space, the method comprising use of a system according to claim 1.

20. A method for improving air quality and reducing the risk of infection to a human being or animal in an enclosed space, the method comprising steps:
a) providing a tubular housing with an inlet to draw in air from the enclosed space and an outlet to release air back into the enclosed space;
b) optionally producing ozone in a first section of the tubular housing, proximate the inlet, to disinfect air therein;
c) producing hydroxyl ions in a second section of the tubular housing downstream of the first section, to disinfect air therein.

21. (canceled)

22. (canceled)
